(19) United States
(12) Patent Application Publication
(10) Pub. No.: US 2008/0091102 A1
(43) Pub. Date: Apr. 17, 2008

(54) Image Processing Apparatus and Image Processing Method

(76) Inventors: Hisato Maeda, Toyoake-shi (JP); Noriyasu Yamaki, Nishinomiya (JP)

Correspondence Address: FOLEY & LARDNER LLP, P.O. BOX 80278, SAN DIEGO, CA, 92138-0278, US

(21) Appl. No.: 11/857,307

(22) Filed: Sep. 18, 2007

Related U.S. Application Data
(60) Provisional application No. 60/845,507, filed on Sep. 19, 2006.

(51) Int. Cl. A61B 6/00

(52) U.S. Cl. 600/436

(57) ABSTRACT

An image processing apparatus (1) comprises: a projection image data input unit (10) for inputting nuclear medicine image data; a Butterworth filter (12) for filtering the nuclear medicine image data; and a cutoff frequency setting unit (20) for setting a cutoff frequency of the Butterworth filter (12). The cutoff frequency setting unit (20) has an optimum cutoff frequency table (22) storing a relation between counts in nuclear medicine image data and an optimum cutoff frequency. The image processing apparatus (1) determines counts in the nuclear medicine image data, determines based on the optimum cutoff frequency table (22) a cutoff frequency corresponding to the determined counts, and sets a cutoff frequency of the Butterworth filter (12) at the determined cutoff frequency.

### CROSS-REFERENCES TO RELATED APPLICATIONS

This application claims priority to U.S. Provisional Application No. 60/845,507, filed Sep. 19, 2006, which is hereby incorporated by reference in its entirety, including all tables, drawings, figures and claims.

### FIELD OF THE INVENTION

The present invention relates to a method of setting a condition of a filter used for image processing of a nuclear medicine image, and to an image processing apparatus and program for carrying out the method. In particular, the invention relates to a method of determining a cutoff frequency of a Butterworth filter used for image processing of a nuclear medicine image, and to an image processing apparatus and program for carrying out the method. Nuclear medicine images to which the invention is applied include an image provided by positron emission tomography (PET) and an image provided by single photon emission computed tomography (SPECT).

### BACKGROUND OF THE INVENTION

Nuclear medicine images, typified by a PET image and a SPECT image, are effective for a diagnosis of heart failure, cancer, and other various diseases. These images can be obtained by giving a subject a drug labeled with a specific radioisotope (hereinafter referred to as a “radiopharmaceutical”), detecting gamma rays emitted directly or indirectly from the drug by means of a dedicated camera, and reconstructing. Here, acquired image data includes noise caused by statistical fluctuations. In general, creating a reconstruction image without removing noise from acquired image data cannot provide an image that can be used in diagnosis. In order to obtain a good image that can be used in diagnosis, it is required to remove noise included in image data, prior to an image reconstruction process.

As methods of removing noise included in image data, there are known a filtering process using a Butterworth filter, a Gaussian filter, or the like, a smoothing process, and the like. The most relied-upon and clinically widely-used method of these methods is a method in which image data is filtered with a Butterworth filter. In this method, image data is multiplied by a filter function that is determined by an order and a cutoff frequency.

### BRIEF SUMMARY OF THE INVENTION

Removing noise using a Butterworth filter requires that an order and a cutoff frequency be set. Of these parameters, a cutoff frequency is known to greatly affect image quality of a reconstruction image. Butterworth filtering without using an appropriate cutoff frequency may cause an incorrect diagnosis result.

Conventionally, a cutoff frequency of a Butterworth filter would be set based on the experience of an operator. That is, an optimum value would be determined by image processing being repeated a plurality of times with a cutoff frequency being varied. However, this method has a problem that image quality of a nuclear medicine image to be obtained largely depends on the experience and skill of an operator. For image processing, a method is desired in which a cutoff frequency can be set uniquely, but such a method is hitherto unknown.

A purpose of the invention made in view of the above-mentioned background is to provide an image processing apparatus, an image processing method, and a program that can perform filtering with an optimum cutoff frequency being set automatically.

The present inventors have made intensive study, and as a result, have found that there is a correlation between counts in nuclear medicine image data and an optimum cutoff frequency at which noise is minimized. Based on this finding, the inventors have completed the invention in which an optimum cutoff frequency is set for a Butterworth filter.

In one aspect of the invention, an image processing apparatus for processing nuclear medicine image data comprises: an image input unit for inputting nuclear medicine image data; a Butterworth filter for filtering the nuclear medicine image data; and a cutoff frequency setting unit for setting a cutoff frequency of the Butterworth filter, where the cutoff frequency setting unit has an optimum cutoff frequency table storing a relation between counts in nuclear medicine image data and an optimum cutoff frequency which is a Butterworth filter cutoff frequency at which noise in nuclear medicine image data is minimized by filtering, and where the image processing apparatus determines counts in the nuclear medicine image data, determines based on the optimum cutoff frequency table a cutoff frequency corresponding to the determined counts, and sets a cutoff frequency of the Butterworth filter at the determined cutoff frequency.

In another aspect of the invention, the optimum cutoff frequency table may store a relation between total counts in a region of interest (ROI) and the optimum cutoff frequency.

In another aspect of the invention, the optimum cutoff frequency table may store a relation between counts per unit area in a region of interest (ROI) and the optimum cutoff frequency.

In another aspect of the invention, the optimum cutoff frequency table may have a different table for each targeted part of nuclear medicine image data, and the image processing apparatus may have a part information input unit for inputting information on a targeted part of the nuclear medicine image data, where the cutoff frequency setting unit may choose a table according to a part inputted by the part information input unit, and may determine an optimum cutoff frequency based on the chosen table.

In another aspect of the invention, a table generation apparatus for generating a table used for setting a Butterworth filter cutoff frequency in image processing of nuclear medicine image data comprises: an image acquisition unit for acquiring: ideal nuclear medicine image data of a phantom obtained by imaging the phantom with counts being increased so that noise can be ignored; and nuclear medicine image data obtained by imaging the phantom under condition of a number of counts being a prescribed number; and an optimum cutoff frequency calculator for filtering nuclear medicine image data acquired by the image acquisition unit with a Butterworth filter cutoff frequency being varied, and for determining an optimum cutoff frequency at which an error of the nuclear medicine image data with respect to the ideal nuclear medicine image data is minimized, where the image acquisition unit successively acquires nuclear medicine image data under condition of counts being varied, the optimum cutoff frequency calculator determines an optimum cutoff frequency for nuclear medicine image data concerned, and thereby an optimum cutoff frequency is determined for each number of counts.

In another aspect of the invention, a table generation apparatus for generating a table used for setting a Butterworth filter cutoff frequency in image processing of nuclear medicine image data comprises: an anatomical image generator for generating a prescribed anatomical image in a virtual space; a nuclear medicine image calculator for determining by calculation: ideal noiseless nuclear medicine image data that will be obtained if the anatomical image is imaged; and nuclear medicine image data that will be obtained if the anatomical image is imaged under condition of a number of counts being a prescribed number; and an optimum cutoff frequency calculator for filtering nuclear medicine image data obtained under condition of a number of counts being a prescribed number, with a Butterworth filter cutoff frequency being varied, and for determining an optimum cutoff frequency at which an error of the nuclear medicine image data with respect to the ideal noiseless nuclear medicine image data is minimized, where the nuclear medicine image calculator successively calculates nuclear medicine image data under condition of counts being varied, the optimum cutoff frequency calculator determines an optimum cutoff frequency for nuclear medicine image data concerned, and thereby an optimum cutoff frequency is determined for each number of counts.

In another aspect of the invention, an image processing method comprises the steps of: (1) inputting nuclear medicine image data; (2) determining counts in the nuclear medicine image data, determining a cutoff frequency corresponding to the determined counts, based on an optimum cutoff frequency table storing a relation between counts in nuclear medicine image data and an optimum cutoff frequency, and setting a Butterworth filter cutoff frequency at the determined cutoff frequency, where the optimum cutoff frequency being a Butterworth filter cutoff frequency at which noise in nuclear medicine image data is minimized by filtering; and (3) filtering the nuclear medicine image data using a Butterworth filter whose cutoff frequency is set in the step 2.

In another aspect of the invention, the optimum cutoff frequency table may store a relation between total counts in a region of interest (ROI) and the optimum cutoff frequency.

In another aspect of the invention, the optimum cutoff frequency table may store a relation between counts per unit area in a region of interest (ROI) and the optimum cutoff frequency.

In another aspect of the invention, the optimum cutoff frequency table may have a different table for each targeted part of nuclear medicine image data, and the image processing method may have a step of inputting information on a targeted part of the nuclear medicine image data, where, in the step of setting a cutoff frequency for a Butterworth filter, a table may be chosen according to the inputted part, and an optimum cutoff frequency may be determined based on the chosen table.

In another aspect of the invention, the optimum cutoff frequency table may be generated by the steps of: (1) acquiring ideal nuclear medicine image data of a phantom by imaging the phantom with counts being increased so that noise can be ignored; (2) acquiring nuclear medicine image data by imaging the phantom under condition of a number of counts being a prescribed number; (3) filtering nuclear medicine image data acquired in the step 2 with a Butterworth filter cutoff frequency being varied, and determining an optimum cutoff frequency at which an error of the nuclear medicine image data with respect to the ideal nuclear medicine image data is minimized; and (4) repeating the steps 2 and 3 under condition of counts of the step 2 being varied, and thereby determining an optimum cutoff frequency for each number of counts.

In another aspect of the invention, the optimum cutoff frequency table may be generated by the steps of: (1) generating a prescribed anatomical image in a virtual space; (2) determining by calculation ideal noiseless nuclear medicine image data that will be obtained if the anatomical image is imaged; (3) determining by calculation nuclear medicine image data that will be obtained if the anatomical image is imaged under condition of a number of counts being a prescribed number; (4) filtering nuclear medicine image data determined in the step 3 with a Butterworth filter cutoff frequency being varied, and determining an optimum cutoff frequency at which an error of the nuclear medicine image data with respect to the ideal noiseless nuclear medicine image data is minimized; and (5) repeating the steps 3 and 4 under condition of counts of the step 3 being varied, and thereby determining an optimum cutoff frequency for each number of counts.

In another aspect of the invention, a table generation method for generating a table used for setting a Butterworth filter cutoff frequency in image processing of nuclear medicine image data comprises the steps of: (1) acquiring ideal nuclear medicine image data of a phantom by imaging the phantom with counts being increased so that noise can be ignored; (2) acquiring nuclear medicine image data by imaging the phantom under condition of a number of counts being a prescribed number; (3) filtering nuclear medicine image data acquired in the step 2 with a Butterworth filter cutoff frequency being varied, and determining an optimum cutoff frequency at which an error of the nuclear medicine image data with respect to the ideal nuclear medicine image data is minimized; and (4) repeating the steps 2 and 3 under condition of counts of the step 2 being varied, and thereby determining an optimum cutoff frequency for each number of counts.

In another aspect of the invention, a table generation method for generating a table used for setting a Butterworth filter cutoff frequency in image processing of nuclear medicine image data comprises the steps of: (1) generating a prescribed anatomical image in a virtual space; (2) determining by calculation ideal noiseless nuclear medicine image data that will be obtained if the anatomical image is imaged; (3) determining by calculation nuclear medicine image data that will be obtained if the anatomical image is imaged under condition of a number of counts being a prescribed number; (4) filtering nuclear medicine image data determined in the step 3 with a Butterworth filter cutoff frequency being varied, and determining an optimum cutoff frequency at which an error of the nuclear medicine image data with respect to the ideal noiseless nuclear medicine image data is minimized; and (5) repeating the steps 3 and 4 under condition of counts of the step 3 being varied, and thereby determining an optimum cutoff frequency for each number of counts.

In another aspect of the invention, a program causes a computer to execute the steps of: (1) inputting nuclear medicine image data; (2) determining counts in the nuclear medicine image data, determining a cutoff frequency corresponding to the determined counts, based on an optimum cutoff frequency table storing a relation between counts in nuclear medicine image data and an optimum cutoff frequency, and setting a Butterworth filter cutoff frequency at the determined cutoff frequency, where the optimum cutoff frequency being a Butterworth filter cutoff frequency at which noise in nuclear medicine image data is minimized by filtering; and (3) filtering the nuclear medicine image data using a Butterworth filter whose cutoff frequency is set in the step 2.

In another aspect of the invention, the optimum cutoff frequency table may store a relation between total counts in a region of interest (ROI) and the optimum cutoff frequency.

In another aspect of the invention, the optimum cutoff frequency table may store a relation between counts per unit area in a region of interest (ROI) and the optimum cutoff frequency.

In another aspect of the invention, the optimum cutoff frequency table may have a different table for each targeted part of nuclear medicine image data, and the program may have a step of inputting information on a targeted part of the nuclear medicine image data, where, in the step of setting a cutoff frequency for a Butterworth filter, a table may be chosen according to the inputted part, and an optimum cutoff frequency may be determined based on the chosen table.

In another aspect of the invention, the optimum cutoff frequency table may be generated by the steps of: (1) acquiring ideal nuclear medicine image data of a phantom obtained by imaging the phantom with counts being increased so that noise can be ignored; (2) acquiring nuclear medicine image data obtained by imaging the phantom under condition of a number of counts being a prescribed number; (3) filtering nuclear medicine image data acquired in the step 2 with a Butterworth filter cutoff frequency being varied, and determining an optimum cutoff frequency at which an error of the nuclear medicine image data is minimized with respect to the ideal nuclear medicine image data is minimized; and (4) repeating the steps 2 and 3 under condition of counts of the step 2 being varied, and thereby determining an optimum cutoff frequency for each number of counts.

In another aspect of the invention, the optimum cutoff frequency table may be generated by the steps of: (1) generating a prescribed anatomical image in a virtual space; (2) determining by calculation ideal noiseless nuclear medicine image data that will be obtained if the anatomical image is imaged; (3) determining by calculation nuclear medicine image data that will be obtained if the anatomical image is imaged under condition of a number of counts being a prescribed number; (4) filtering nuclear medicine image data determined in the step 3 with a Butterworth filter cutoff frequency being varied, and determining an optimum cutoff frequency at which an error of the nuclear medicine image data with respect to the ideal noiseless nuclear medicine image data is minimized; and (5) repeating the steps 3 and 4 under condition of counts of the step 3 being varied, and thereby determining an optimum cutoff frequency for each number of counts.

In another aspect of the invention, a program for generating a table used for setting a Butterworth filter cutoff frequency in image processing of nuclear medicine image data causes a computer to execute the steps of: (1) acquiring ideal nuclear medicine image data of a phantom obtained by imaging the phantom with counts being increased so that noise can be ignored; (2) acquiring nuclear medicine image data obtained by imaging the phantom under condition of a number of counts being a prescribed number; (3) filtering nuclear medicine image data acquired in the step 2 with a Butterworth filter cutoff frequency being varied, and determining an optimum cutoff frequency at which an error of the nuclear medicine image data with respect to the ideal nuclear medicine image data is minimized; and (4) repeating the steps 2 and 3 under condition of counts of the step 2 being varied, and thereby determining an optimum cutoff frequency for each number of counts.

In another aspect of the invention, a program for generating a table used for setting a Butterworth filter cutoff frequency in image processing of nuclear medicine image data causes a computer to execute the steps of: (1) generating a prescribed anatomical image in a virtual space; (2) determining by calculation ideal noiseless nuclear medicine image data that will be obtained if the anatomical image is imaged; (3) determining by calculation nuclear medicine image data that will be obtained if the anatomical image is imaged under condition of a number of counts being a prescribed number; (4) filtering nuclear medicine image data determined in the step 3 with a Butterworth filter cutoff frequency being varied, and determining an optimum cutoff frequency at which an error of the nuclear medicine image data with respect to the ideal noiseless nuclear medicine image data is minimized; and (5) repeating the steps 3 and 4 under condition of counts of the step 3 being varied, and thereby determining an optimum cutoff frequency for each number of counts.

### BRIEF DESCRIPTION OF THE DRAWINGS

FIG. 1 shows a configuration of an image processing apparatus of an embodiment;

FIG. 2 shows an example of data stored in an optimum cutoff frequency table;

FIG. 3 shows an operation of the image processing apparatus of the embodiment;

FIG. 4 shows an operation to generate the optimum cutoff frequency table;

FIG. 5 shows an operation to calculate an optimum cutoff frequency;

FIG. 6 shows a configuration of a program of the embodiment;

FIG. 7 shows an example of a graph on which MSE values are plotted against cutoff frequencies f_{c};

FIG. 8 shows an example of a relation between counts in the cardiac region and optimum cutoff frequencies f_{c}, as to a nuclear medicine image of a breast;

FIG. 9 shows an example of a reconstruction image of a nuclear medicine image of a breast to which a ^{99m}Tc-tetrofosmin injection is given;

FIG. 10 is a graph on which cutoff frequencies obtained by visual evaluation are superimposed on an optimum cutoff frequency table for a nuclear medicine image of a breast, as to data acquired by using an LEHR collimator; and

FIG. 11 is a graph on which cutoff frequencies obtained by visual evaluation are superimposed on an optimum cutoff frequency table for a nuclear medicine image of a breast, as to data acquired by using an LEGP collimator.

### DETAILED DESCRIPTION OF THE INVENTION

[Configuration of the Image Processing Apparatus]

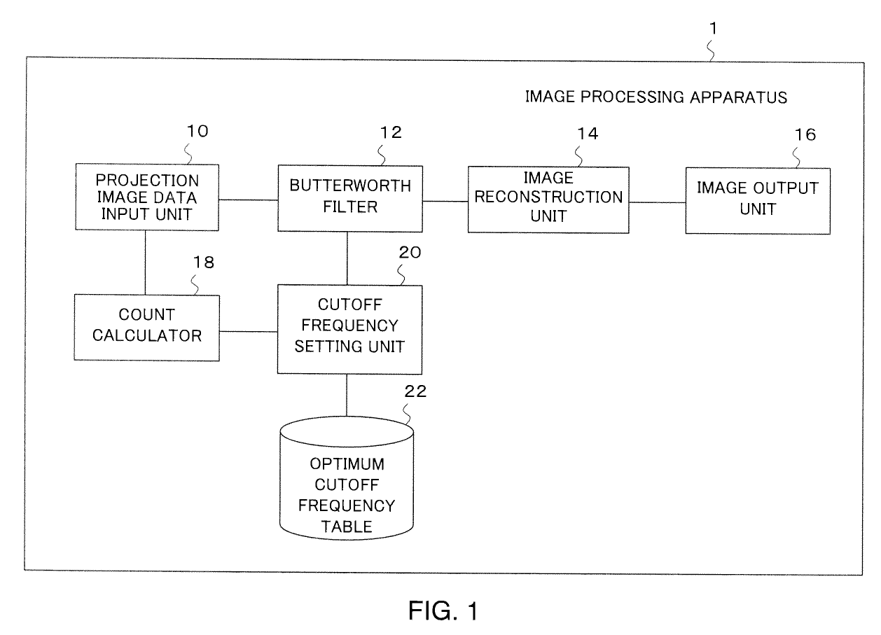

Now, an image processing apparatus of an embodiment of the invention will be described with reference to the drawings. FIG. 1 shows a configuration of an image processing apparatus 1 of the embodiment. The image processing apparatus 1 comprises: a projection image data input unit 10 for inputting projection image data imaged by a PET or other imaging apparatus; a Butterworth filter 12 for filtering an image inputted to the projection image data input unit 10; an image reconstruction unit 14 for reconstructing a filtered image to generate a three-dimensional image; and an image output unit 16 for outputting an image generated by the image reconstruction unit 14.

As a configuration for setting a cutoff frequency of the Butterworth filter 12, the image processing apparatus 1 comprises a count calculator 18, a cutoff frequency setting unit 20, and an optimum cutoff frequency table 22. The count calculator 18 calculates counts in projection image data inputted to the projection image data input unit 10. In the embodiment, count calculator 18 calculates total counts in a region of interest (ROI) of projection image data. The cutoff frequency setting unit 20 reads from the optimum cutoff frequency table 22 an optimum cutoff frequency corresponding to counts in a projection image calculated by the count calculator 18, and set a cutoff frequency of the Butterworth filter 12 at the read cutoff frequency.

The optimum cutoff frequency table 22 stores a relation between counts in nuclear medicine image data and an optimum cutoff frequency at which noise is minimized. An optimum cutoff frequency is a cutoff frequency at which noise in nuclear medicine image data is minimized by filtering. In other words, the cutoff frequency minimizes an error of filtered nuclear medicine image data with respect to an ideal image.

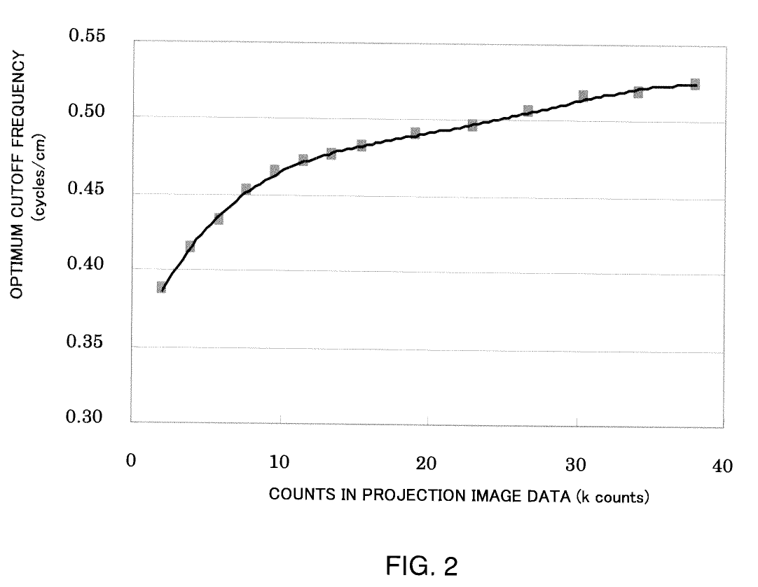

FIG. 2 shows an example of data stored in the optimum cutoff frequency table 22. The horizontal axis represents counts in projection image data, and the vertical axis represents an optimum cutoff frequency. The optimum cutoff frequency table 22 stores a relation between counts and an optimum cutoff frequency as a function representing a curve. In a case where a relation between counts and an optimum cutoff frequency is given in the form of an approximation curve as shown in FIG. 2, an optimum cutoff frequency can be determined by substituting the number of counts into an equation representing the curve.

The optimum cutoff frequency table 22 may store as discrete data a plurality of representative counts and the corresponding optimum cutoff frequencies in association with each other. In this case, as for counts not existing in the optimum cutoff frequency table 22, an optimum cutoff frequency is determined by interpolating neighboring data.

The optimum cutoff frequency table 22 can be obtained by objectively evaluating the difference between an ideal image being under very little influence of noise (under substantially no influence of noise) and a nuclear medicine image. Data obtained by a simulation using a phantom is used for this evaluation. As for the phantom, a Hoffman Phantom or other commercially available phantom may be used, or a numerical phantom constructed in a virtual space may be used. Using a numerical phantom allows an ideal image to be obtained easily. A method of generating data to be stored in the optimum cutoff frequency table 22 will be described later.

[Operation of the Image Processing Apparatus]

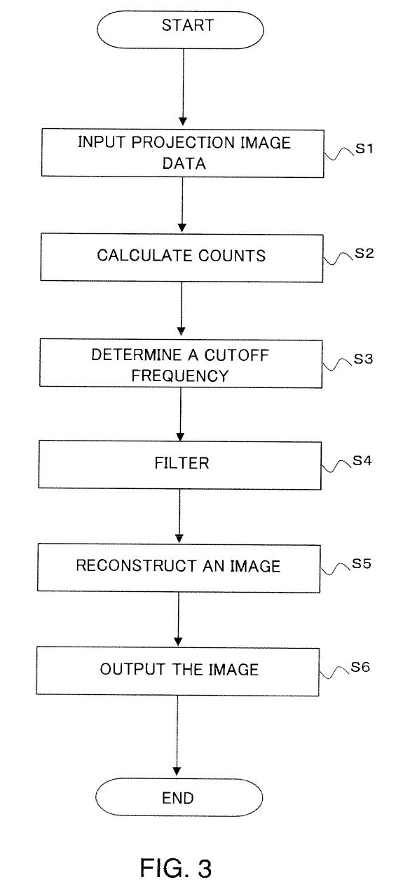

FIG. 3 is a flowchart showing an operation of the image processing apparatus 1 First, the image processing apparatus 1 accepts an input of projection image data (S1). The image processing apparatus 1 then calculates counts in the inputted projection image data (S2). The image processing apparatus 1 reads from the optimum cutoff frequency table 22 a cutoff frequency corresponding to the inputted counts, and set the Butterworth filter 12 at the read cutoff frequency (S3). The image processing apparatus 1 filters the projection image data at the set cutoff frequency (S4). Subsequently, the image processing apparatus 1 reconstructs the filtered projection image data to generate a reconstruction image (S5), and outputs the generated reconstruction image (S6).

There has been described the image processing apparatus 1 of the embodiment of the invention. The image processing apparatus 1 of the embodiment has the optimum cutoff frequency table 22 storing a relation between counts and an optimum cutoff frequency, reads from the optimum cutoff frequency table 22 a cutoff frequency associated with counts, and thereby determines an optimum cutoff frequency. Performing Butterworth filtering using this cutoff frequency can provide nuclear medicine image data with reduced noise, and can provide an appropriate diagnosis.

[Generation of the Optimum Cutoff Frequency Table]

A method of generating the optimum cutoff frequency table 22 (see FIGS. 1 and 2), which the image processing apparatus 1 has, will be described next. The optimum cutoff frequency table 22 can be generated by using various methods. In the following, one example of generation of the optimum cutoff frequency table 22 will be described with reference to drawings.

In the embodiment, instead of using a physical phantom, a numerical phantom constructed in a virtual space is used to generate the optimum cutoff frequency table 22. This allows an ideal reconstruction image being under very little influence of noise (hereinafter referred to as an “ideal image”) to be determined by calculation, and therefore an optimum cutoff frequency at which an error can be determined in a short time.

(General Flow of the Table Generation Process)

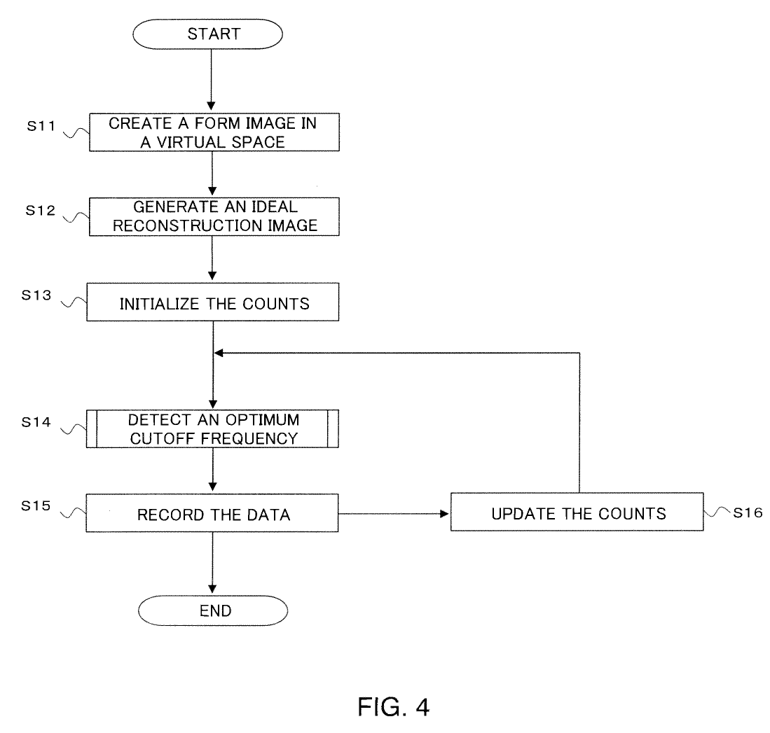

FIG. 4 shows an example of the general flow of a process of generating the optimum cutoff frequency table 22. The image processing apparatus 1 creates an anatomical image of a targeted part in a virtual space (step S11). Based on an anatomical image created in this step, cutoff frequencies of the Butterworth filter 12 are evaluated.

The creation of an anatomical image (step S11) will be described here in detail. The image processing apparatus 1 creates a contour image of a tomogram of a targeted part, and obtains an anatomical image by distributing, on the contour image, radioactivity concentration ratios that differ from one organ to another. A contour image is a tomogram of a targeted part represented by a contour of an organ included in a cross section. As for creation of a contour image, a method can be used in which a contour image is created with reference to anatomical image data, such as CT and MRI, or to standard organ data, such as the Talairach brain atlas. An anatomical image can be created, for example, by drawing directly on a computer display, using a mouse or other input means. The image processing apparatus 1 distributes, on the created contour image, each organ's radioactivity ratio assumed for a targeted radiopharmaceutical, and thereby obtains anatomical image data.

The image processing apparatus 1 then generates an ideal image of the anatomical image (step S12). The ideal image to be generated here is a reconstruction image that does not include noise caused by statistical fluctuations. In the embodiment, the image processing apparatus 1 obtains reconstruction images with counts being varied. The image processing apparatus 1 determines optimum cutoff frequency for each count, and stores the determined optimum cutoff frequency in the optimum cutoff frequency table 22 in association with counts.

The image processing apparatus 1 sets in a computer an initial value of counts in a nuclear medicine image (step S13), and detects an optimum cutoff frequency for the counts (step S14). As for an initial value of counts, arbitrary counts can be used within a range in which nuclear medicine image data is usually acquired. Upon detecting an optimum cutoff frequency, the image processing apparatus 1 records the detected optimum cutoff frequency in association with the counts (step S15). The image processing apparatus 1 changes the counts (step S16). As for the changing process for the counts, a method can preferably be adopted in which a prescribed numerical value is successively added to the counts. A method may also be adopted in which a plurality of counts are stored in advance and the stored counts are read successively.

The image processing apparatus 1 detects an optimum cutoff frequency for the changed counts (step S14). By repeating the steps S14 to S16 in this way, the image processing apparatus 1 determines an optimum cutoff frequency for each number of counts, and records the determined optimum cutoff frequency. The relation between counts and an optimum cutoff frequency is recorded in the optimum cutoff frequency table 22 for a range wide enough to cover a range for which it is likely to be implemented in clinical practice. The image processing apparatus 1 may allow a data plot representing the relation between counts and an optimum cutoff frequency to be the optimum cutoff frequency table 22, or may allow information representing an approximation curve determined from the data plot concerned to be the optimum cutoff frequency table 22. It is preferable to use the approximation curve because an optimum cutoff frequency can also be easily determined for counts other than the counts for which optimum cutoff frequencies were determined. The approximation curve can be obtained from a data plot of optimum cutoff frequencies by using a least squares method or other publicly known methods.

(Process of Calculating an Optimum Cutoff Frequency)

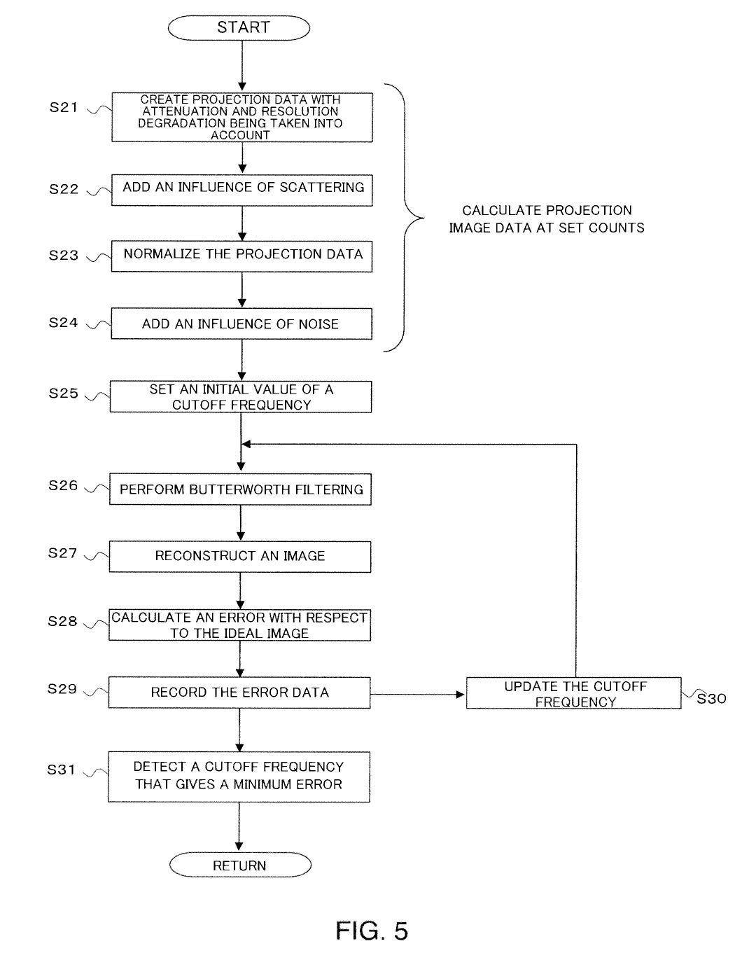

The process of detecting an optimum cutoff frequency at a time when a prescribed number of counts is set (step S14, see FIG. 4) will be described in detail with reference to FIG. 5.

FIG. 5 shows an example of a process of detecting an optimum cutoff frequency corresponding to counts. By calculation, the image processing apparatus 1 determines projection image data of a numerical phantom generated in the step S11 shown in FIG. 4 and imaged at counts set in the step S13 or S16 (steps S21 to S24).

Using the created anatomical image, the image processing apparatus 1 creates projection image data with attenuation and resolution degradation of gamma rays being taken into account (step S21). In a preferred configuration, projection image data with attenuation being taken into account can be determined by the following method.

A ratio At(a,θ), by which a gamma-ray beam emitted from a given part a of an anatomical image is attenuated by body tissue until the beam is detected by a detector at a projection angle θ, is expressed by the following equation (1):
At(a,θ)=exp{−(μ₁·l_{1θ}·μ₂·l_{2θ}+ . . . +μₙ·l_{nθ})} (1)
where μ₁, μ₂, . . . , μₙ represent the linear attenuation coefficients of tissue 1, tissue 2, . . . , tissue n, respectively, and l_{1θ}, l_{2θ}, . . . , l_{nθ} represent the distances over which a gamma-ray beam emitted from the pixel a passes through tissue 1, tissue 2, . . . , tissue n, respectively, until it reaches the detector. This calculation is performed for all pixels and for all projection angles, and projection image data originating from the corresponding pixel is multiplied by the result. Thereby, projection image data with an influence of attenuation being taken into account, I(a,θ), can be obtained.

The image processing apparatus 1 then adds an influence of resolution degradation to the above-mentioned projection image data with an influence of attenuation being taken into account, I(a,θ), and thereby turns it to be projection image data with attenuation and resolution degradation of gamma rays being taken into account. Resolution degradation intended here is an image blur that depends on the distance from a radiation source. The addition of an influence of resolution degradation can be performed with the following manner.

A so-called point spread function is assumed which represents a ratio by which a gamma-ray beam emitted from a given part enters a detector surface at a point with a given coordinate. This point spread function is commonly known to be well approximated by using a Gaussian function (Noriyasu Yamaki et al., Simultaneous spatial resolution correction in SPECT reconstruction using OS-EM algorithm, Japanese Journal of Medical Physics, 24-2, 2004, 61-71). In the embodiment, the point spread function f(p) is assumed to be a Gaussian function expressed by the following (2):

$$f ( p ) = \frac{1}{\sqrt{2 π} \times σ} \times {ⅇ}^{\frac{{( p - μ )}^{2}}{2 {σ}^{2}}} \qquad ( 2 )$$

where σ is the standard deviation, μ is a coordinate of a point at which a gamma-ray beam emitted from a given part vertically enters the detector surface, and p is a coordinate of a given point on the detector surface. It is known that σ, which is an index representing a spread, depends on the distance from a radiation source to a detector. For an object collimator, a relation between the distance from the radiation source to the detector and σ is examined in advance as to an image using a point source, and projection image data with attenuation and resolution degradation of gamma rays being taken into account, g(a,θ), is obtained by using each l(a,θ) and f (p) substituted with a corresponding to each a and θ, in accordance with an equation (3):
g(a,θ)=I(a,θ) [symbol] (f(p) (3)
where in the equation (3), [symbol] represents convolution. This calculation is performed for all pixels and for all θ, and thereby projection image data with attenuation and resolution degradation being taken into account can be obtained.

After obtaining projection image data with attenuation and resolution degradation being taken into account, the image processing apparatus 1 adds an influence of scattering to the projection image data concerned (step S22). In the above example, the scattering component can be given by the following equation (4):
g_{SC}(a,θ)=[g(a,θ)S(a)]·K (4)
where g_{SC}(a,θ) is the scattering component, S(a) is a scattering function, and K is a scattering constant. Corresponding g_{SC}(a,θ) and g(a,θ) are added together, and thereby an influence of scattering is added to the projection image data.

The scattering function and the scattering constant can be determined by using a Monte Carlo method or the like and, in order to reduce the calculation load, they can be approximated by using a result determined by the following manner. In concrete terms, a result determined by using actually acquired data can be used for the scattering function. That is, data is acquired in accordance with a triple energy window method (T. Ichihara et al., Compton scatter compensation using the triple-energy window method for single- and dual-isotope SPECT, J. Nucl. Med., 34, 1993, 2216-2221), and scatter-corrected main window data is convoluted with various Gaussian functions. With use of the results, MSEs are calculated with respect to sub-window data used for scatter correction, and a Gaussian function with which the MSE is minimized is determined to be the scattering function. As for the scattering constant, the ratio between counts in scatter-corrected main window image and total counts in sub-window images on the high energy side and low energy side is determined from each projection image data, and the mean value of the results is determined to be the scattering constant.

After completing the addition of an influence of scattering, the image processing apparatus 1 normalizes the value of projection image data originating from each pixel, using the value of total counts in the nuclear medicine image assumed in the simulation (step S23). For example, in a case where the very value of a ratio representing a radioactivity concentration ratio is distributed to each pixel on the anatomical image created in the step S11 (see FIG. 4), each projection image data to which an influence of scattering is added is multiplied by the total counts concerned.

After completion of the data normalization, an influence of noise is added to the projection image data (step S24). In a preferred configuration, the addition of an influence of noise is in accordance with the following way of thinking and method. It is commonly known that the probability of gamma rays being emitted from a radionuclide is in accordance with a Poisson distribution. If so, the probability of a given pixel's number of counts being N, P(N), is given by the following equation (5):

$$P ( N ) = \frac{{λ}^{N} \cdot exp ( - λ )}{N !} \qquad ( 5 )$$

where λ is a median, and N is the number of counts corresponding to each pixel. The addition of an influence of noise is done by substituting for λ the value of counts in each pixel of normalized projection image data and by calculating in accordance with the equation (5). This is performed for all projection image data, and thereby the addition of an influence of noise is complete. With the process so far, the image processing apparatus 1 determines projection image data of a numerical phantom.

If ideal projection image data including no noise is to be determined, projection image data is determined without executing the step S24 which is for adding an influence of noise. When ideal projection image data is to be reconstructed (S12, see FIG. 4), an ideal reconstruction image (ideal image) is generated based on ideal projection image data including no noise.

After determining projection image data for a set number of counts, the image processing apparatus 1 sets an arbitrary initial value of a cutoff frequency (step S25), and performs Butterworth filtering using this cutoff frequency (step S26).

For the Butterworth filtering, a method is adopted in which a two-dimensional Fourier transform is applied to projection image data for development in frequency space; then a Butterworth filter BF(f) given by the following equation (6) is applied thereto; and further a two-dimensional inverse Fourier transform is applied thereto to transform back to real space:

$$BF ( f ) = \frac{1}{1 + {( \frac{f}{{f}_{c}} )}^{n}} \qquad ( 6 )$$

where f is a frequency, f_{c} is a cutoff frequency, and n is an order. An arbitrary value which is usually used in nuclear medicine image filtering can be used for the order n and, for example, it can be a value from 5 to 15.

After completion of the Butterworth filtering, image reconstruction is carried out (step S27). For reconstruction of projection image data, a publicly known method can be used, such as the FBP algorithm (T. F. Budinger, G. T. Gullberg, Three-dimensional reconstruction in nuclear medicine emission imaging, IEEE Trans. Nucl. Sci. NS-21, 1974, 2-20) and the OS-EM algorithm (H. M. Hudson et al., Accelerated image reconstruction using ordered subsets of projection data, IEEE Trans. Med. Imag. 13, 1994, 601-609). Performing the above series of operations can provide a reconstruction image of a numerical phantom (hereinafter referred to as a “numerical phantom image”).

After obtaining a numerical phantom image corresponding to the set number of counts, the image processing apparatus 1 calculates an error of the numerical phantom image with respect to the ideal image (step S28). Values calculated by using various equations can be used as the error. For example, a mean square error, MSE, given by the following equation (7) can be used:

$$M S E = \frac{{\sum }_{n = 1}^{slice} {\sum }_{x = 1}^{Xmatix} {\sum }_{y = 1}^{Ymatrix} {( {Ideal}_{n} ( x , y ) - {Image}_{n} ( x , y ) )}^{2}}{slice \times Xmatrix \times Ymatrix} \qquad ( 7 )$$

where Idealₙ(x, y) is the signal intensity of the ideal image for slice number n at coordinates (x, y), Image, (x, y) is the signal intensity of the numerical phantom image for slice number n at coordinates (x, y), slice is the number of slices, and Xmatrix and Ymatrix are the numbers of matrices in the directions of the horizontal axis (x-axis) and the vertical axis (y-axis), respectively. By calculating using this equation (7), an error of the numerical phantom image of prescribed counts can be determined with respect to the ideal image.

After an error of the numerical phantom image is determined with respect to the ideal image, the value of the determined error is recorded on a recording medium as error data (step S29). The value of the cutoff frequency is then changed (step S30), and the process from the step S26 to the step S29 is repeated. In a preferred configuration, the cutoff frequency can be updated by adding a prescribed numerical value to the cutoff frequency. A method may also be adopted in which a plurality of cutoff frequencies for which error data is to be calculated are stored; and the stored cutoff frequencies are read successively. The cutoff frequency is changed, the process from the step S26 to the step S29 is repeated, and data is stored, the data being of a range wide enough to detect the minimum value of the error. With use of the series of data, the value of a cutoff frequency that gives a minimum MSE is detected (step S31), so that an optimum cutoff frequency value for the given counts is obtained.

[Program]

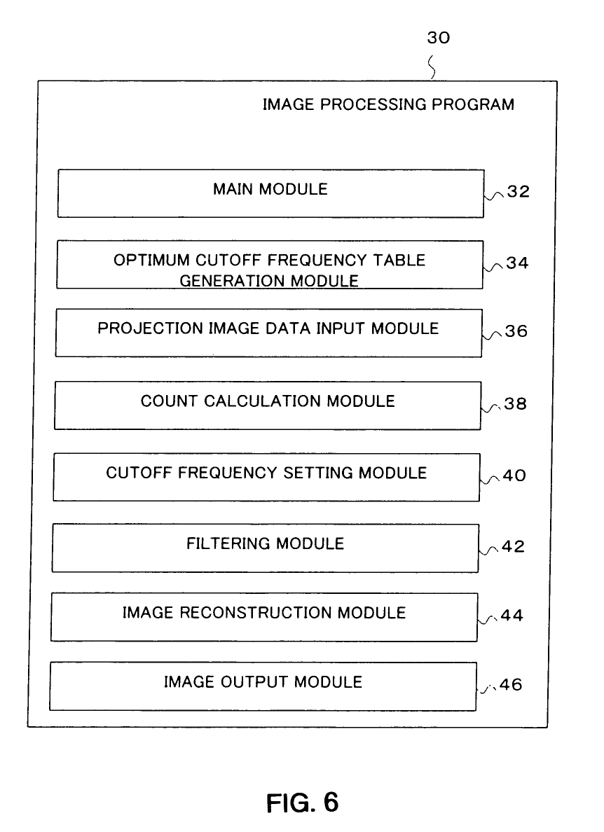

FIG. 6 shows a program of the embodiment of the invention. An image processing program 30 of the embodiment comprises: a main module 32 for unifying a process; an optimum cutoff frequency table generation module 34; a projection image data input module 36; a count calculation module 38; a cutoff frequency setting module 40; a filtering module 42; an image reconstruction module 44; and an image output module 46.

The main module 32 is a module for controlling the execution of: the optimum cutoff frequency table generation module 34; the projection image data input module 36; the count calculation module 38; the cutoff frequency setting module 40; the filtering module 42; the image reconstruction module 44; and the image output module 46.

By causing the computer to execute, the optimum cutoff frequency table generation module 34 generates the optimum cutoff frequency table 22 shown in FIG. 1 in accordance with the procedure described in FIGS. 4 and 5.

By causing the computer to execute, the projection image data input module 36 realizes the function of the projection image data input unit 10 shown in FIG. 1. This allows the computer to accept an input of projection image data on an object to be processed.

By causing the computer to execute, the count calculation module 38 realizes the function of the count calculator 18 shown in FIG. 1. This allows the computer to calculate counts in projection image data.

By causing the computer to execute, the cutoff frequency setting module 40 realizes the function of the cutoff frequency setting unit 20 shown in FIG. 1. This allows the computer to read from the optimum cutoff frequency table 22 a cutoff frequency associated with calculated counts, and to set the Butterworth filter 12 at the read cutoff frequency.

By causing the computer to execute, the filtering module 42 performs filtering using the Butterworth filter 12 on inputted projection image data at a cutoff frequency set by the cutoff frequency setting module 40.

By causing the computer to execute, the image reconstruction module 44 realizes the function of the image reconstruction unit 14 shown in FIG. 1. The computer reconstructs filtered projection image data and generates a three-dimensional image.

By causing the computer to execute, the image output module 46 realizes the function of the image output unit 16 shown in FIG. 1. The computer outputs a nuclear medicine image reconstructed by the image reconstruction module 44.

As a recording medium for recording the program of the embodiment, there is a hard disk, a flexible disk, a CD-ROM, a DVD, or other recording media, such as a ROM, or semiconductor memories. A recording medium in which the program 30 is stored is inserted into a reader provided on the computer, and the program 30 thereby becomes accessible to the computer, so that the program 30 concerned allows the computer to operate as the image processing apparatus 1.

The program 30 may be provided via a network as a computer data signal superimposed on a carrier wave. In this case, the program 30 is received by a communications device provided on the computer to be stored in a memory also provided on the computer, and is thereby executed by the computer.

While there have been described the image processing apparatus, image processing method, and program of the invention in detail with reference to an embodiment, the invention is not limited to the above-described embodiment.

In the above-described embodiment, a plurality of optimum cutoff frequency tables may be provided according to the type of collimator to be used for a data acquisition, to acquisition conditions including a matrix size, to a part to be imaged and its size, and the like. This allows the filtering process to be performed at an appropriate cutoff frequency in accordance with acquisition conditions and the like.

In the above-described embodiment, for example, counts per unit area in an ROI set on a contour image of a targeted part or the like may be used.

In the above-described embodiment, an example has been described in which the optimum cutoff frequency table 22 is determined by using an anatomical image constructed in a virtual space. Alternatively, the optimum cutoff frequency table 22 may be determined based on a result of actually imaging a physical phantom. In this case, an ideal image can be imaged by increasing counts sufficiently. Sufficiently increased counts allow noise caused by statistical fluctuations to be ignored.

In the above-described embodiment, an example has been described in which counts in nuclear medicine image data is calculated based on an inputted nuclear medicine image. Alternatively, a configuration may be adopted in which counts acquired by a PET or other imaging apparatus are inputted.

### WORKING EXAMPLE

In the following, the invention will be described in further detail with reference to a working example, but the invention is not limited to the contents of the description. In the working example, an optimum cutoff frequency table generated according to the above-described embodiment was compared with cutoff frequencies obtained by visual evaluation.

(Generation of an Optimum Cutoff Frequency Table)

To begin with, a procedure in which an optimum cutoff frequency table was generated will be described.

(1) Creation of a Numerical Phantom Image and an Ideal Image

An image space having 33 slices with matrix size of 128×128 (hereinafter referred to as a “slice plane”) was prepared in a computer memory (3.2 mm/pixel). A contour image was created for each slice plane with reference to a breast MRI image. For the created contour image, radioactivity concentration ratios of 30, 10, and 1 were inputted to pixels corresponding to myocardium, liver, and soft tissue (muscle and fat), respectively.

The process of the above-described step S21 was then performed on the above image, and projection image data was obtained with an influence of attenuation and resolution degradation being added. The addition of an influence of resolution degradation was made with the assumption that an LEHR collimator (Low Energy High Resolution collimator) was used and that an LEGP collimator (Low Energy General Purpose collimator) was used, and the radius of rotation for each collimator was set at 23 cm (a usually-used average radius of rotation). In the process, the linear attenuation coefficient of the heart and soft tissue including muscle and fat was set at 0.15 cm⁻¹, the linear attenuation coefficient of the lung was set at 0.03 cm⁻¹, and the linear attenuation coefficient of a bone was set at 0.30 cm⁻¹ (^{99m}Tc formulation was intended).

The process of the above-described step S22 was then applied to the above projection image data, and an influence of scattering wad added thereto. In this process, the scattering function and the scattering constant were determined in accordance with a triple energy window method (T. Ichihara et al., Compton scatter compensation using the triple-energy window method for single- and dual-isotope SPECT, J. Nucl. Med., 34, 1993, 2216-2221) intended for an image for a case where a ^{99m}Tc formulation was given. The calculation using the triple energy window method was performed on eight examples of data, and mean values of the results were used as values of the scattering function and the scattering constant.

The process of the above-described step S23 was then applied to the above projection image data, and the data was normalized by using total counts. In concrete terms, a process was performed in which each component comprising the projection image data was multiplied by counts. Total counts used for the normalization were 5, 10, 15, 20, 25, 30, 35, 40, 50, 70, and 100 (kcounts/projection) for both LEGP and LEHR collimators.

The process of the above-described step S24 was performed on this normalized projection image data, and an influence of noise was added thereto. In addition, the process of the step S26 was performed on this projection image data, and the filtering process was performed. A 360-degree back projection was performed on the obtained projection image data to perform an image reconstruction of the step S27, and a numerical phantom image was obtained. The cutoff frequency of the filtering process, f_{c}, was set between 0.25 cycles/cm and 0.65 cycles/cm at intervals of 0.002 cycles/cm.

(2) Creation of Optimum Cutoff Frequency Table Data

With use of the numerical phantom image and of an ideal image created by using the same value of counts as that of the numerical phantom image concerned, a mean square error, MSE, was calculated by using the following equation (7).

$$M S E = \frac{{\sum }_{n = 1}^{slice} {\sum }_{x = 1}^{Xmatix} {\sum }_{y = 1}^{Ymatrix} {( {Ideal}_{n} ( x , y ) - {Image}_{n} ( x , y ) )}^{2}}{slice \times Xmatrix \times Ymatrix} \qquad ( 7 )$$

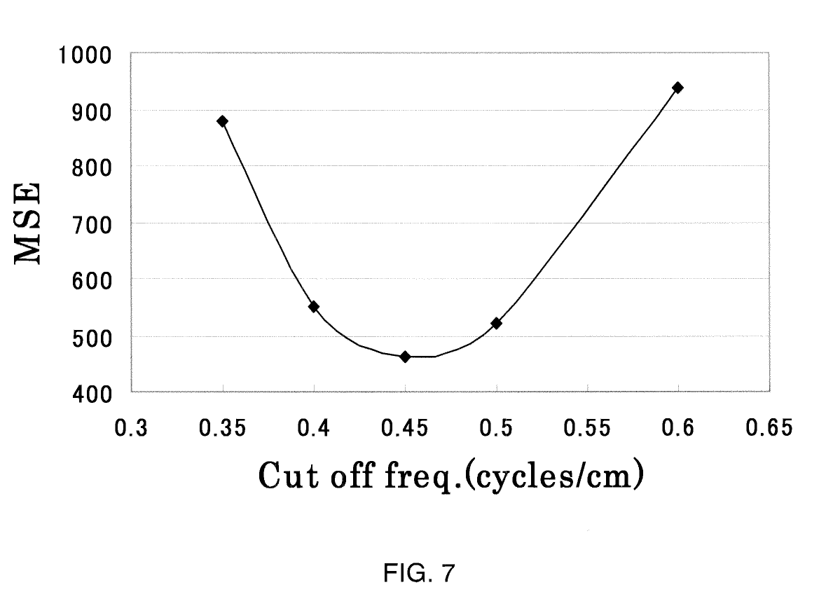

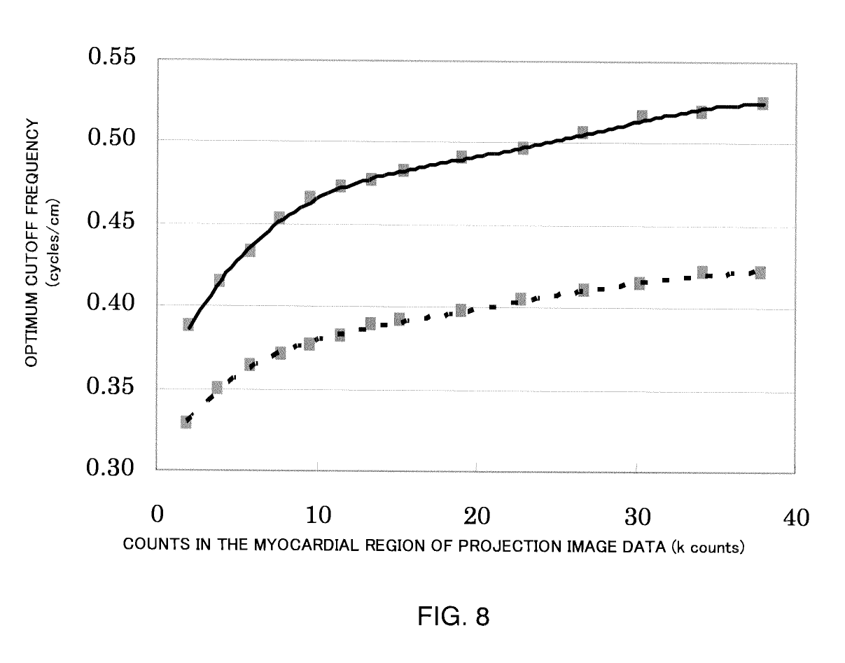

A graph was created in which MSE values were plotted against cutoff frequencies f_{c} for a nuclear medicine image of a prescribed number of counts (FIG. 7). With use of the result, a cutoff frequency f_{c} that gave a minimum MSE was determined to be an optimum cutoff frequency f_{c} for this number of counts. This operation was performed with the number of counts being varied, and obtained optimum cutoff frequencies f_{c} were plotted. Based on the plotted data, an approximation curve was created by means of a least squares method, and was determined to be an optimum cutoff frequency table for nuclear medicine images of breasts (FIG. 8). The solid line is an approximation curve corresponding to an optimum cutoff frequency table for the LEHR collimator, and the broken line is that for the LEGP collimator.

(Cutoff Frequency by Visual Evaluation)

There were obtained: 28 samples of projection image data of myocardial SPECT in which a thallium (²⁰¹Tl) chloride injection was given, the data having been acquired by using a GP collimator; and 11 and 28 samples of projection image data of myocardial SPECT in which a ^{99m}Tc-tetrofosmin injection was given, the data having been acquired by using a GP collimator and an HR collimator, respectively. For each projection image data, an ROI was set in the myocardial part, and the number of counts in the part concerned was determined.

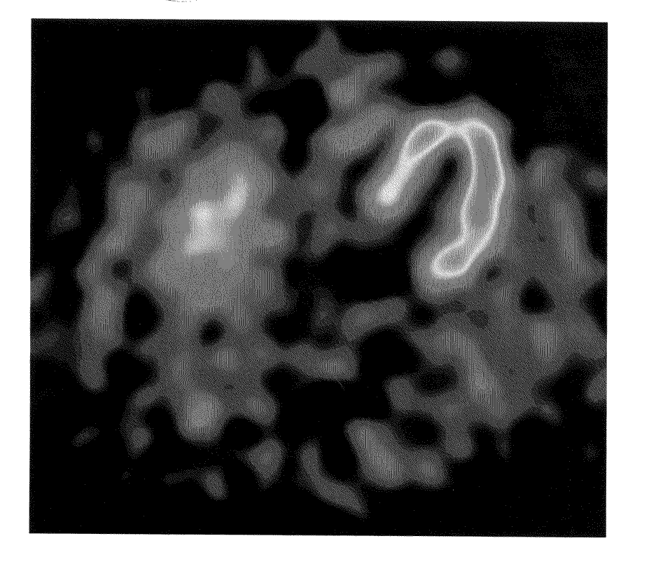

After that, for each projection image data, a Butterworth filtering using a given cutoff frequency was performed and an image reconstruction was performed. This operation was repeated with the cutoff frequency being varied, a best reconstructed image was chosen, and the cutoff frequency concerned was recorded. An example of the reconstruction images is shown in FIG. 9. FIG. 9 is a reconstruction image which was obtained under condition of the number of counts being 23.8 kcounts (myocardial part: 3.0 kcounts) and the cutoff frequency f_{c} at the time of reconstruction being 0.43 cycles/cm.

A graph was created in which the determined cutoff frequencies were plotted against myocardial counts, and it was superimposed on the approximation curve defined by the optimum cutoff frequency table for a nuclear medicine image of a breast.

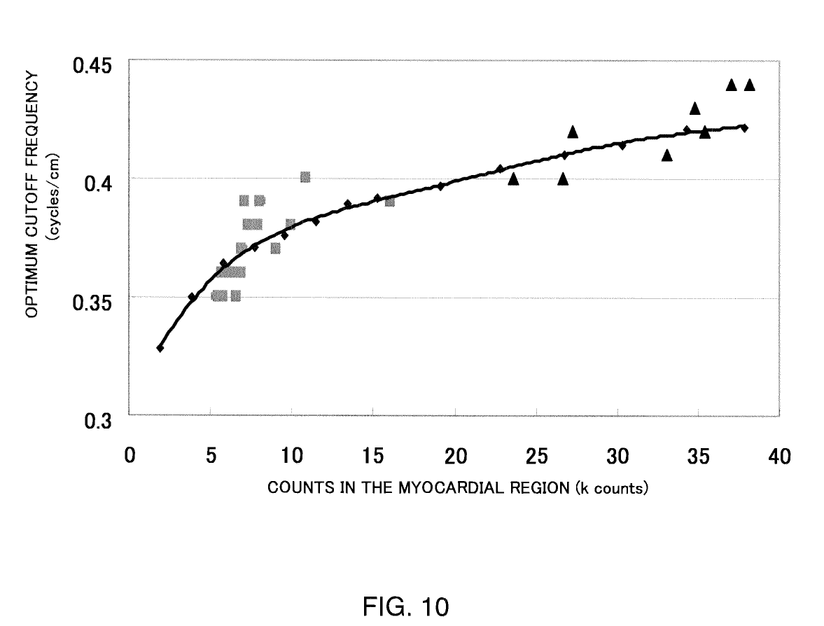

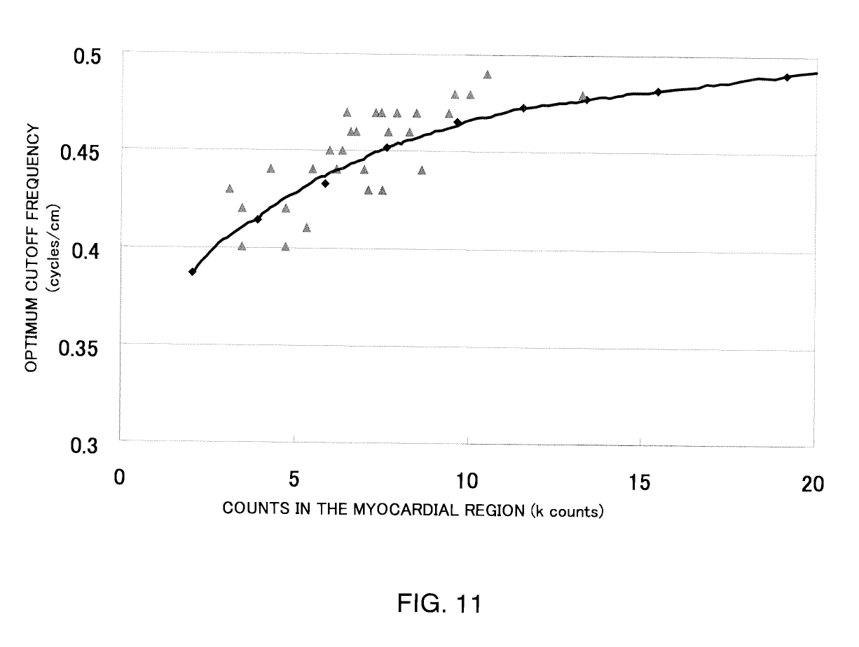

FIGS. 10 and 11 show results of the comparison between the optimum cutoff frequency table and the cutoff frequencies obtained by visual evaluation. FIG. 10 is a graph on which the cutoff frequencies obtained by visual evaluation are superimposed on the approximation curve defined by the optimum cutoff frequency table for a nuclear medicine image of a breast, as to data acquired by using the LEHR collimator. The cutoff frequencies obtained by visual evaluation are indicated by ▪ for the case in which a thallium (²⁰¹Tl) chloride injection was given, and by ▴ for the case in which a ^{99m}Tc-tetrofosmin injection was given. FIG. 11 is a graph on which the cutoff frequencies obtained by visual evaluation are superimposed on the approximation curve defined by the optimum cutoff frequency table for a nuclear medicine image of a breast, as to data acquired by using the LEGP collimator. The cutoff frequencies f_{c} obtained by visual evaluation are indicated by ▴ for the case in which a ^{99m}Tc-tetrofosmin injection was given.

As shown in FIGS. 10 and 11, it was shown that the cutoff frequencies obtained by visual evaluation agreed well with the optimum cutoff frequency table determined by the method of the invention.

The invention is useful as an image processing apparatus for diagnostic images of PET, SPECT, and the like.

The documents referenced herein are incorporated herein by reference.

1. An image processing apparatus for processing nuclear medicine image data, the image processing apparatus comprising:
an image input unit for inputting nuclear medicine image data;
a Butterworth filter for filtering the nuclear medicine image data; and
a cutoff frequency setting unit for setting a cutoff frequency of the Butterworth filter,
wherein the cutoff frequency setting unit has an optimum cutoff frequency table storing a relation between counts in nuclear medicine image data and an optimum cutoff frequency which is a Butterworth filter cutoff frequency at which noise in nuclear medicine image data is minimized by filtering,
and wherein the image processing apparatus determines counts in the nuclear medicine image data, determines based on the optimum cutoff frequency table a cutoff frequency corresponding to the determined counts, and sets a cutoff frequency of the Butterworth filter at the determined cutoff frequency.

2. The image processing apparatus according to claim 1, wherein the optimum cutoff frequency table stores a relation between total counts in a region of interest (ROI) and the optimum cutoff frequency.

3. The image processing apparatus according to claim 1, wherein the optimum cutoff frequency table stores a relation between counts per unit area in a region of interest (ROI) and the optimum cutoff frequency.

4. The image processing apparatus according to claim 1, wherein the optimum cutoff frequency table has a different table for each targeted part of nuclear medicine image data,
the image processing apparatus having a part information input unit for inputting information on a targeted part of the nuclear medicine image data,
and wherein the cutoff frequency setting unit chooses a table according to a part inputted by the part information input unit, and determines an optimum cutoff frequency based on the chosen table.

5. A table generation apparatus for generating a table used for setting a Butterworth filter cutoff frequency in image processing of nuclear medicine image data, the table generation apparatus comprising:
an image acquisition unit for acquiring: ideal nuclear medicine image data of a phantom obtained by imaging the phantom with counts being increased so that noise can be ignored; and nuclear medicine image data obtained by imaging the phantom under condition of a number of counts being a prescribed number; and
an optimum cutoff frequency calculator for filtering nuclear medicine image data acquired by the image acquisition unit with a Butterworth filter cutoff frequency being varied, and for determining an optimum cutoff frequency at which an error of the nuclear medicine image data with respect to the ideal nuclear medicine image data is minimized,
wherein the image acquisition unit successively acquires nuclear medicine image data under condition of counts being varied, the optimum cutoff frequency calculator determines an optimum cutoff frequency for nuclear medicine image data concerned, and thereby an optimum cutoff frequency is determined for each number of counts.

6. A table generation apparatus for generating a table used for setting a Butterworth filter cutoff frequency in image processing of nuclear medicine image data, the table generation apparatus comprising:
an anatomical image generator for generating a prescribed anatomical image in a virtual space;
a nuclear medicine image calculator for determining by calculation: ideal noiseless nuclear medicine image data that will be obtained if the anatomical image is imaged; and nuclear medicine image data that will be obtained if the anatomical image is imaged under condition of a number of counts being a prescribed number; and
an optimum cutoff frequency calculator for filtering nuclear medicine image data obtained under condition of a number of counts being a prescribed number, with a Butterworth filter cutoff frequency being varied, and for determining an optimum cutoff frequency at which an error of the nuclear medicine image data with respect to the ideal noiseless nuclear medicine image data is minimized,
wherein the nuclear medicine image calculator successively calculates nuclear medicine image data under the condition of counts being varied, the optimum cutoff frequency calculator determines an optimum cutoff frequency for nuclear medicine image data concerned, and thereby an optimum cutoff frequency is determined for each number of counts.

7. An image processing method comprising the steps of:
(1) inputting nuclear medicine image data;
(2) determining counts in the nuclear medicine image data, determining a cutoff frequency corresponding to the determined counts, based on an optimum cutoff frequency table storing a relation between counts in nuclear medicine image data and an optimum cutoff frequency, and setting a Butterworth filter cutoff frequency at the determined cutoff frequency, wherein the optimum cutoff frequency being a Butterworth filter cutoff frequency at which noise in nuclear medicine image data is minimized by filtering; and
(3) filtering the nuclear medicine image data using a Butterworth filter whose cutoff frequency is set in the step 2.

8. The image processing method according to claim 7, wherein the optimum cutoff frequency table stores a relation between total counts in a region of interest (ROI) and the optimum cutoff frequency.

9. The image processing method according to claim 7, wherein the optimum cutoff frequency table stores a relation between counts per unit area in a region of interest (ROI) and the optimum cutoff frequency.

10. The image processing method according to claim 7, wherein the optimum cutoff frequency table has a different table for each targeted part of nuclear medicine image data,
the image processing method having a step of inputting information on a targeted part of the nuclear medicine image data,
and wherein, in the step of setting a cutoff frequency for a Butterworth filter, a table is chosen according to the inputted part, and an optimum cutoff frequency is determined based on the chosen table.

11. The image processing method according to claim 7, wherein the optimum cutoff frequency table is generated by the steps of:
(1) acquiring ideal nuclear medicine image data of a phantom by imaging the phantom with counts being increased so that noise can be ignored;
(2) acquiring nuclear medicine image data by imaging the phantom under condition of a number of counts being a prescribed number;
(3) filtering nuclear medicine image data acquired in the step 2 with a Butterworth filter cutoff frequency being varied, and determining an optimum cutoff frequency at which an error of the nuclear medicine image data with respect to the ideal nuclear medicine image data is minimized; and
(4) repeating the steps 2 and 3 under condition of counts of the step 2 being varied, and thereby determining an optimum cutoff frequency for each number of counts.

12. The image processing method according to claim 7, wherein the optimum cutoff frequency table is generated by the steps of:
(1) generating a prescribed anatomical image in a virtual space;
(2) determining by calculation ideal noiseless nuclear medicine image data that will be obtained if the anatomical image is imaged;
(3) determining by calculation nuclear medicine image data that will be obtained if the anatomical image is imaged under condition of a number of counts being a prescribed number;
(4) filtering nuclear medicine image data determined in the step 3 with a Butterworth filter cutoff frequency being varied, and determining an optimum cutoff frequency at which an error of the nuclear medicine image data with respect to the ideal noiseless nuclear medicine image data is minimized; and
(5) repeating the steps 3 and 4 under condition of counts of the step 3 being varied, and thereby determining an optimum cutoff frequency for each number of counts.

13. A table generation method for generating a table used for setting a Butterworth filter cutoff frequency in image processing of nuclear medicine image data, the table generation method comprising the steps of:
(1) acquiring ideal nuclear medicine image data of a phantom by imaging the phantom with counts being increased so that noise can be ignored;
(2) acquiring nuclear medicine image data by imaging the phantom under condition of a number of counts being a prescribed number;
(3) filtering nuclear medicine image data acquired in the step 2 with a Butterworth filter cutoff frequency being varied, and determining an optimum cutoff frequency at which an error of the nuclear medicine image data with respect to the ideal nuclear medicine image data is minimized; and
(4) repeating the steps 2 and 3 under condition of counts of the step 2 being varied, and thereby determining an optimum cutoff frequency for each number of counts.

14. A table generation method for generating a table used for setting a Butterworth filter cutoff frequency in image processing of nuclear medicine image data, the table generation method comprising the steps of:
(1) generating a prescribed anatomical image in a virtual space;
(2) determining by calculation ideal noiseless nuclear medicine image data that will be obtained if the anatomical image is imaged;
(3) determining by calculation nuclear medicine image data that will be obtained if the anatomical image is imaged under condition of a number of counts being a prescribed number;
(4) filtering nuclear medicine image data determined in the step 3 with a Butterworth filter cutoff frequency being varied, and determining an optimum cutoff frequency at which an error of the nuclear medicine image data with respect to the ideal noiseless nuclear medicine image data is minimized; and
(5) repeating the steps 3 and 4 under condition of counts of the step 3 being varied, and thereby determining an optimum cutoff frequency for each number of counts.

15. A program for causing a computer to execute the steps of:
(1) inputting nuclear medicine image data;
(2) determining counts in the nuclear medicine image data, determining a cutoff frequency corresponding to the determined counts, based on an optimum cutoff frequency table storing a relation between counts in nuclear medicine image data and an optimum cutoff frequency, and setting a Butterworth filter cutoff frequency at the determined cutoff frequency, wherein the optimum cutoff frequency being a Butterworth filter cutoff frequency at which noise in nuclear medicine image data is minimized by filtering; and
(3) filtering the nuclear medicine image data using a Butterworth filter whose cutoff frequency is set in the step 2.

16. The program according to claim 15, wherein the optimum cutoff frequency table stores a relation between total counts in a region of interest (ROI) and the optimum cutoff frequency.

17. The program according to claim 15, wherein the optimum cutoff frequency table stores a relation between counts per unit area in a region of interest (ROI) and the optimum cutoff frequency.

18. The program according to claim 15, wherein the optimum cutoff frequency table has a different table for each targeted part of nuclear medicine image data,
the program having a step of inputting information on a targeted part of the nuclear medicine image data,
and wherein, in the step of setting a cutoff frequency for a Butterworth filter, a table is chosen according to the inputted part, and an optimum cutoff frequency is determined based on the chosen table.

19. The program according to claim 15, wherein the optimum cutoff frequency table is generated by the steps of:
(1) acquiring ideal nuclear medicine image data of a phantom obtained by imaging the phantom with counts being increased so that noise can be ignored;
(2) acquiring nuclear medicine image data obtained by imaging the phantom under condition of a number of counts being a prescribed number;
(3) filtering nuclear medicine image data acquired in the step 2 with a Butterworth filter cutoff frequency being varied, and determining an optimum cutoff frequency at which an error of the nuclear medicine image data with respect to the ideal nuclear medicine image data is minimized; and
(4) repeating the steps 2 and 3 under condition of counts of the step 2 being varied, and thereby determining an optimum cutoff frequency for each number of counts.

20. The program according to claim 15, wherein the optimum cutoff frequency table is generated by the steps of:
(1) generating a prescribed anatomical image in a virtual space;
(2) determining by calculation ideal noiseless nuclear medicine image data that will be obtained if the anatomical image is imaged;
(3) determining by calculation nuclear medicine image data that will be obtained if the anatomical image is imaged under condition of a number of counts being a prescribed number;
(4) filtering nuclear medicine image data determined in the step 3 with a Butterworth filter cutoff frequency being varied, and determining an optimum cutoff frequency at which an error of the nuclear medicine image data with respect to the ideal noiseless nuclear medicine image data is minimized; and
(5) repeating the steps 3 and 4 under condition of counts of the step 3 being varied, and thereby determining an optimum cutoff frequency for each number of counts.

21. A program for generating a table used for setting a Butterworth filter cutoff frequency in image processing of nuclear medicine image data, the program causing a computer to execute the steps of:
(1) acquiring ideal nuclear medicine image data of a phantom obtained by imaging the phantom with counts being increased so that noise can be ignored;
(2) acquiring nuclear medicine image data obtained by imaging the phantom under condition of a number of counts being a prescribed number;
(3) filtering nuclear medicine image data acquired in the step 2 with a Butterworth filter cutoff frequency being varied, and determining an optimum cutoff frequency at which an error of the nuclear medicine image data with respect to the ideal nuclear medicine image data is minimized; and
(4) repeating the steps 2 and 3 under condition of counts of the step 2 being varied, and thereby determining an optimum cutoff frequency for each number of counts.

22. A program for generating a table used for setting a Butterworth filter cutoff frequency in image processing of nuclear medicine image data, the program causing a computer to execute the steps of:
(1) generating a prescribed anatomical image in a virtual space;
(2) determining by calculation ideal noiseless nuclear medicine image data that will be obtained if the anatomical image is imaged;
(3) determining by calculation nuclear medicine image data that will be obtained if the anatomical image is imaged under condition of a number of counts being a prescribed number;
(4) filtering nuclear medicine image data determined in the step 3 with a Butterworth filter cutoff frequency being varied, and determining an optimum cutoff frequency at which an error of the nuclear medicine image data with respect to the ideal noiseless nuclear medicine image data is minimized; and
(5) repeating the steps 3 and 4 under condition of counts of the step 3 being varied, and thereby determining an optimum cutoff frequency for each number of counts.
